(19) United States
(12) Patent Application Publication
(10) Pub. No.: US 2016/0250277 A1
(43) Pub. Date: Sep. 1, 2016

(54) PERIPHERAL KAPPA OPIOID RECEPTOR AGONISTS FOR PREVENTING, INHIBITING OR TREATING NAUSEA AND VOMITING

(71) Applicant: CARA THERAPEUTICS, INC., Shelton, CT (US)

(72) Inventors: Derek T. CHALMERS, Riverside, CT (US); James B. JONES, Metuchen, NJ (US)

(73) Assignee: Cara Therapeutics, Inc., Shelton, CT (US)

(21) Appl. No.: 15/032,351

(22) PCT Filed: Oct. 27, 2014
(86) PCT No.: PCT/US14/62320

§ 371 Date: Apr. 27, 2016

Related U.S. Application Data
(60) Provisional application No. 61/896,469, filed on Oct. 28, 2013.

(51) Int. Cl. A61K 38/07; A61K 31/40; A61K 31/485; A61K 9/00

(52) U.S. Cl. CPC A61K 38/07; A61K 9/0019; A61K 31/40; A61K 31/485

(57) ABSTRACT

A method for preventing, inhibiting or treating nausea and/or vomiting in a mammalian subject, the method comprising administering an effective amount of a peripherally-restricted kappa opioid receptor agonist to the subject. The nausea and/or vomiting can be associated with use of an opioid, such as morphine or fentanyl. The peripherally-restricted kappa opioid receptor agonist can be an L-amino acid-containing peptide, a D-amino acid-containing peptide, or a synthetic peptide amide, such as for instance, D-Phe-D-Phe-D-Leu-D-Lys-[ω(4-aminopiperidine-4-carboxylic acid)]-OH (CR845).

Strong mu opioid analgesics, such as morphine, fentanyl, or hydromorphone, are mainstays of pain treatment in the immediate postoperative period, and are used as part of a multimodal analgesic approach. However, the use of strong mu opioid analgesics is associated with an array of unwanted and serious side effects, including postoperative opioid-induced respiratory depression, or POIRD, postoperative nausea and vomiting, or PONV, and opioid-induced bowel dysfunction, or OBD, which contributes to the severity of postoperative ileus, or POI. The incidence of POIRD may be as high as 29 percent, can occur unexpectedly in even the healthiest of patients, and exerts a disproportionately high toll on length of stay and hospital costs due to the significant expenses associated with the treatment of POIRD. PONV occurs in approximately one-third of surgical patients overall, and is an important factor in determining length of stay after surgery, resulting in annual costs in the U.S. in the range of $1 billion. These mu opioid-related adverse events not only significantly increase the cost of care, but also reduce a patient's quality of care and lead to sub-optimal recovery.

In 2005, the FDA announced a requirement for boxed warnings of potential cardiovascular risk for all NSAIDs. The FDA warning related to cardiovascular adverse events associated with NSAIDs and the increased awareness of the risk of liver toxicity associated with high doses of acetaminophen have led to increased use of mu opioid analgesics for the treatment of chronic pain. However, the use of mu opioid analgesics carries significant additional risks. Chronic opioid use causes patients to develop tolerance for the opioid, which results in the patient needing increasing opioid doses to achieve the same level of pain relief. For the most commonly prescribed analgesic combination products, the need for increasing doses to achieve the same level of pain relief means exposure to increasing amounts of NSAIDs or acetaminophen, which carry the risks attendant to these therapeutics. Moreover, due to their CNS activity, mu opioids produce feelings of euphoria, which can give rise to abuse and addiction. Underlining the severity of this issue, in 2013, the FDA announced class-wide safety labeling changes and new post-market study requirements for all extended-release and long-acting mu opioid analgesics intended to treat pain. In addition, as a result of their potential for misuse, abuse and addiction, currently approved mu opioids are strictly regulated by the United States Drug Enforcement Agency (DEA), under the Controlled Substances Act, which imposes strict registration, record keeping and reporting requirements, security control and restrictions on prescriptions—all of which significantly increase the costs and the liability attendant to prescription opioid analgesics.

### SUMMMARY

The present invention provides a method for preventing, inhibiting or treating nausea and vomiting in a mammalian subject, the method comprising administering an effective amount of a peripherally-restricted kappa opioid receptor agonist to the subject. In one embodiment, the peripherally-restricted kappa opioid receptor agonist includes a peptide. In another embodiment, the peptide includes one or more D-amino acids.

In one embodiment the present invention provides a method for preventing, inhibiting or treating nausea and vomiting in a mammalian subject, the method comprising administering an effective amount of a peripherally-restricted kappa opioid receptor agonist, wherein the peripherally restricted kappa opioid receptor agonist comprises a synthetic peptide amide having the formula:

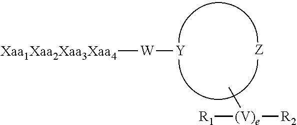

or a stereoisomer, mixture of stereoisomers, prodrug, pharmaceutically acceptable salt, hydrate, solvate, acid salt hydrate, N-oxide or isomorphic crystalline form thereof.

In one embodiment, the residue Xaa₁ is selected from the group consisting of (A)(A′)D-Phe, (A)(A′)(α-Me)D-Phe, D-Tyr, D-Tic, D-tert-leucine, D-neopentylglycine, D-phenylglycine, D-homophenylalanine, and β-(E)D-Ala, wherein each (A) and each (A′) are phenyl ring substituents independently selected from the group consisting of —H, —F, —Cl, —NO₂, —CH₃, —CF₃, —CN, and —CONH₂, and wherein each (E) is independently selected from the group consisting of cyclobutyl, cyclopentyl, cyclohexyl, pyridyl, thienyl and thiazolyl; Xaa₂ is selected from the group consisting of (A)(A′)D-Phe, 3,4-dichloro-D-Phe, (A)(A′)(α-Me)D-Phe, D-1Nal, D-2Nal, D-Tyr, (E)D-Ala and D-Trp; Xaa₃ is selected from the group consisting of D-Nle, D-Phe, (E)D-Ala, D-Leu, (α-Me)D-Leu, D-Hle, D-Val, and D-Met; Xaa₄ is selected from the group consisting of (B)₂D-Arg, (B)₂D-Nar, (B)₂D-Har, ζ-(B)D-Hlys, D-Dap, ε-(B)D-Lys, ε-(B)₂-D-Lys, D-Amf, amidino-D-Amf, γ-(B)₂D-Dbu, δ-(B)₂α-(B′)D-Orn, D-2-amino-3(4-piperidyl)propionic acid, D-2-amino-3(2-aminopyrrolidyl)propionic acid, D-α-amino-β-amidinopropionic acid, α-amino-4-piperidineacetic acid, cis-α,4-diaminocyclohexane acetic acid, trans-α,4-diaminocyclohexaneacetic acid, cis-α-amino-4-methylaminocyclo-hexane acetic acid, trans-α-amino-4-methylaminocyclohexane acetic acid, α-amino-1-amidino-4-piperidineacetic acid, cis-α-amino-4-guanidinocyclohexane acetic acid, and trans-α-amino-4-guanidinocyclohexane acetic acid; wherein each (B) is independently selected from the group consisting of H and C₁-C₄ alkyl, and (B′) is H or (α-Me); W is selected from the group consisting of: Null, provided that when W is null, Y is N; —NH—(CH₂)_{b}— with b equal to zero, 1, 2, 3, 4, 5, or 6; and —NH—(CH₂)_{c}—O— with c equal to 2, or 3, provided that Y is C.

In another embodiment, the moiety

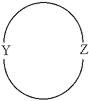

is an optionally substituted 4 to 8-membered heterocyclic ring moiety wherein all ring heteroatoms in said ring moiety are N; wherein Y and Z are each independently C or N; provided that when such ring moiety is a six, seven or eight-membered ring, Y and Z are separated by at least two ring atoms; and provided that when such ring moiety has a single ring heteroatom which is N, then such ring moiety is non-aromatic; V is C₁-C₆ alkyl, and e is zero or 1, wherein when e is zero, then V is null and R₁ and R₂ are directly bonded to the same or different ring atoms; wherein (i) R₁ is selected from the group consisting of —H, —OH, halo, —CF₃, —NH₂, —COOH, C₁-C₆ alkyl, C₁-C₆ alkoxy, amidino, C₁-C₆ alkyl-substituted amidino, aryl, optionally substituted heterocyclyl, Pro-amide, Pro, Gly, Ala, Val, Leu, Ile, Lys, Arg, Orn, Ser, Thr, —CN, —CONH₂, —COR′, —SO₂R′, —CONR′R″, —NHCOR′, OR′ and SO₂NR′R″; wherein said optionally substituted heterocyclyl is optionally singly or doubly substituted with substituents independently selected from the group consisting of C₁-C₆ alkyl, C₁-C₆ alkoxy, oxo, —OH, —Cl, —F, —NH₂, —NO₂, —CN, —COOH, and amidino; wherein R′ and R″ are each independently —H, C₁-C₈ alkyl, aryl, or heterocyclyl or R′ and R″ are combined to form a 4- to 8-membered ring, which ring is optionally singly or doubly substituted with substituents independently selected from the group consisting of C₁-C₆ alkyl, —C₁-C₆ alkoxy, —OH, —Cl, —F, —NH₂, —NO₂, —CN, —COOH and amidino; and R₂ is selected from the group consisting of -H, amidino, singly or doubly C₁-C₆ alkyl-substituted amidino, —CN, —CONH₂, —CONR′R″, —NHCOR′, —SO₂NR′R″ and —COOH; or (ii) R₁ and R₂ taken together can form an optionally substituted 4- to 9-membered heterocyclic monocyclic or bicyclic ring moiety which is bonded to a single ring atom of the Y and Z-containing ring moiety; or (iii) R₁ and R₂ taken together with a single ring atom of the Y and Z-containing ring moiety can form an optionally substituted 4- to 8-membered heterocyclic ring moiety to form a spiro structure; or (iv) R₁ and R₂ taken together with two or more adjacent ring atoms of the Y and Z-containing ring moiety can form an optionally substituted 4- to 9-membered heterocyclic monocyclic or bicyclic ring moiety fused to the Y and Z-containing ring moiety; wherein each of said optionally substituted 4-, 5-, 6,-, 7-, 8- and 9-membered heterocyclic ring moieties comprising R₁ and R₂ is optionally singly or doubly substituted with substituents independently selected from the group consisting of C₁-C₆ alkyl, C₁-C₆ alkoxy, optionally substituted phenyl, oxo, —OH, —Cl, —F, —NH₂, —NO₂, —CN, —COOH, and amidino; provided that when the Y and Z-containing ring moiety is a six or seven membered ring having a single ring heteroatom and e is zero, then R₁ is not —OH, and R₁ and R₂ are not both —H; and provided further that when the Y and Z-containing ring moiety is a six membered ring having two ring heteroatoms, both Y and Z are N and W is null, then —(V)ₑR₁R₂ is attached to a ring atom other than Z; and if e is zero, then R₁ and R₂ are not both —H.

### BRIEF DESCRIPTION OF THE FIGURES

FIG. 1: Efficacy of CR845 in “Chung Model” of neuropathic pain is blocked with Peripheral (Intrapaw) administration of a kappa antagonist (norBNI) in rats. *** denotes p<0.001 compared to vehicle-treated controls (two-way ANOVA). Vehicle or Nor-BNI was administered intraplantarly (0.2 mg) 15 min prior to CR845. Injection (1 mg/kg). N=6 male rats/group, mean±SEM.

FIG. 2: Phase 2b Laparoscopic Hysterectomy—Summed Pain Intensity Difference from 0-24 Hours (SPID₀₋₂₄) following postoperative treatment. *p≦0.05, **p≦0.01;

FIG. 3: Phase 2b Laparoscopic Hysterectomy—Pain Intensity Difference (PID) at specific times relative to postoperative baseline pain intensity. *p≦0.05, **p≦0.01 for CR845/CR845. #p≦0.05 for both Placebo/CR845 and CR845/Placebo. Values represent mean+SEM

FIG. 4: Phase 2b Laparoscopic Hysterectomy—Total Pain Relief Within the first 2 hours (TOTPARO-2) following postoperative treatment. *p<0.05. Values represent mean+SEM.

FIG. 5: Phase 2b Laparoscopic Hysterectomy—Morphine Consumption For 2-24 hours post-treatment in patients. *p≦0.05; Values represent mean+SEM.

FIG. 6: Phase 2b Laparoscopic Hysterectomy—Incidence of opioid-related adverse events over 24 hours. ***p≦0.001; *p<0.05.

FIG. 7: Phase 2b Laparoscopic Hysterectomy—Responder analysis of global evaluation of study medication. **p=0.001

FIG. 8a: Phase 2 Bunionectomy—Summed Pain Intensity Difference from 0-24 hours (SPID₀₋₂₄), 0-36 hours (p SPIDO-36) and 0-48 hours (SPIDO-48) in completer population.

FIG. 8b: Phase 2 Bunionectomy—Summed Pain Intensity Difference from 0-24 hours (SPID0-24), 0-36 hours (SPID0-36) and 0-48 hours (SPID0-48) in mITT Population (Completers plus non-completers). *p≦0.05—One-sided ANOVA with Treatment Group as a Main Effect (mean+/−SEM).

FIG. 9a: Phase 2 Bunionectomy—Pain Intensity Difference relative to baseline in CR845 and placebo completer treatment groups over a 48 hour period. * p≦0.05 (0-36 hours). ** p≦0.01 (0-12 hours).

FIG. 9b: Phase 2 Bunionectomy—Pain Intensity Difference relative to baseline in CR845 and placebo treatment Groups in mITT populations across 48 hours. *p≦0.05 (0-12 hours)

FIG. 10: Phase 2 Bunionectomy—CR845 Suppression of Nausea and Vomiting. *p<0.05

FIG. 11: Phase la Pharmacokinetic profiles of increasing concentrations of CR845 in capsules in human subjects.

### DETAILED DESCRIPTION

Nausea is an unpleasant experience in humans and probably animals. Physiologically, nausea is typically associated with decreased gastric motility and increased tone in the small intestine. Additionally, there is often reverse peristalsis in the proximal small intestine.

Emesis or vomiting is when gastric and often small intestinal contents are propelled up to and out of the mouth. Usually, a deep breath is taken, the glottis is closed and the larynx is raised to open the upper esophageal sphincter. Also, the soft palate is elevated to close off the nasal cavity. The diaphragm is contracted sharply downward to create negative pressure in the thorax, which opens the esophagus and distal esophageal sphincter. Then, simultaneously with downward movement of the diaphragm, the muscles of the abdominal walls contract vigorously, squeezing the stomach and elevating intragastric pressure. The pylorus closes and the esophagus is open and the vomitus is forced out.

In one embodiment the present invention provides a method for preventing, inhibiting or treating nausea and vomiting in a mammalian subject such as a human, the method comprising administering an effective amount of a peripherally-restricted kappa opioid receptor agonist to the subject, wherein the moiety:

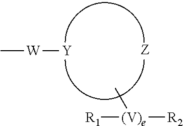

is selected from any one of the following:

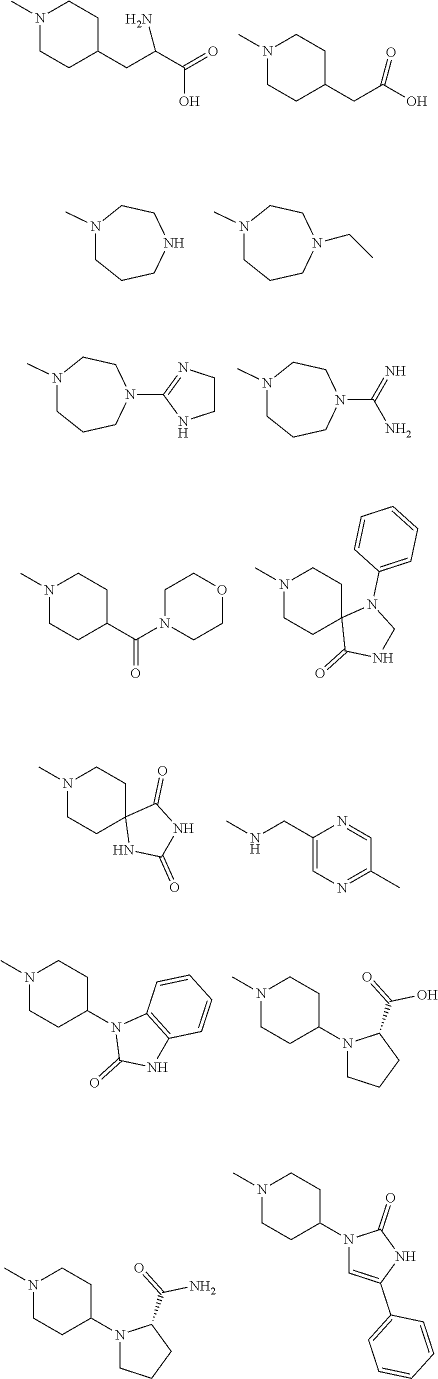

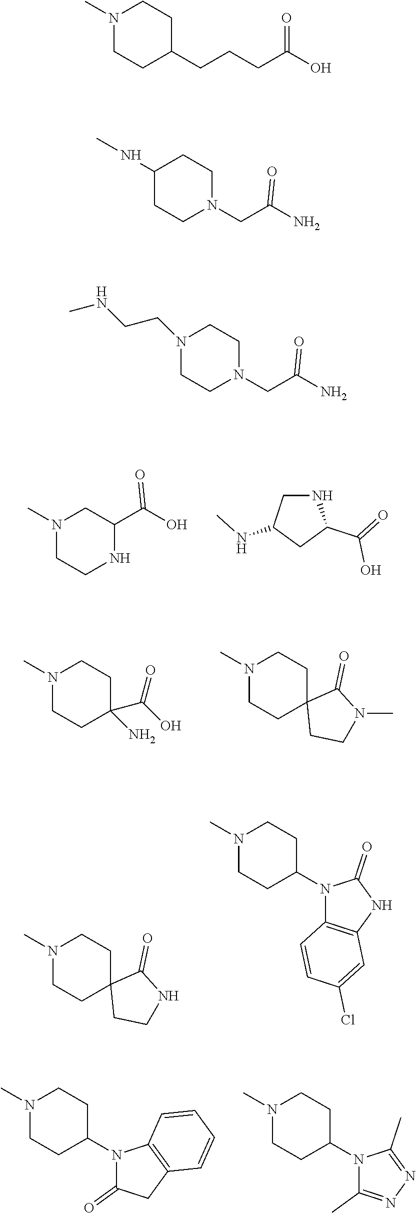

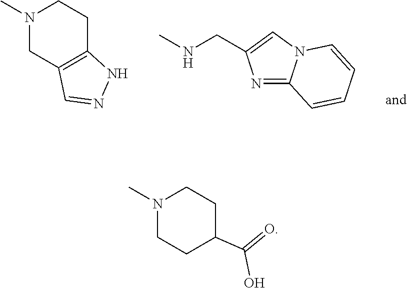

In another embodiment, the invention provides a method for preventing, inhibiting or treating nausea and vomiting in a mammalian subject, the method comprising administering an effective amount of a peripherally-restricted kappa opioid receptor agonist to the subject, wherein the synthetic peptide amide has the structure:

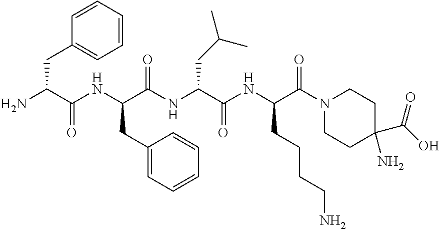 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]

D-Phe-D-Phe-D-Leu-D-Lys-[ω(4-aminopiperidine-4-carboxylic acid)]-OH (also called CR845). The peripherally-restricted kappa opioid receptor agonist can be administered to the subject within 12, 24 or 36 hours prior to, during or within 12, 24 or 36 hours after undergoing a medical procedure. In one embodiment, the medical procedure causes pain, which may be soft tissue pain e.g. muscular pain or visceral pain; or hard tissue pain, e.g. bone pain Kappa opioid receptor agonists and their uses for the prophylaxis, inhibition and treatment of painful and inflammatory diseases, disorders and conditions are described in U.S. Pat. Nos. 7,402,564; 7,713,937; 7,727,963; 7,842,662; 8,217,007; 8,486,894; and 8,536,131, the disclosures of which are hereby incorporated by reference herein in their entireties.

In another embodiment, the invention provides a method for preventing, inhibiting or treating nausea and vomiting in a mammalian subject, wherein the peripherally-restricted kappa opioid receptor agonist is administered to the subject by a route of injection chosen from the following: subcutaneous injection, intravenous injection, intraperitoneal injection, intra-articular injection, and intramuscular injection.

In another embodiment, the peripherally-restricted kappa opioid receptor agonist can be any suitable peripherally-restricted kappa opioid receptor agonist, such as for instance a non-narcotic analgesic, for example, asimadoline (N-[(1S)-2-[(3S)-3 -hydroxypyrrolidin-1-yl]-1-phenylethyl]-N-methyl-2,2-diphenylacetamide), or nalfurafine ((2E)-N-[(5α,6β)-17-(cyclopropylmethyl)-3,14-dihydroxy-4,5-epoxymorphinan-6-yl]-3-(3-furyl)-N-methylacrylamide).

In another embodiment, the invention provides a method for preventing, inhibiting or treating nausea and vomiting in a human, by administering a peripherally-restricted kappa opioid receptor agonist to the human, wherein the nausea and/or vomiting occurs within 48 hours after administration of at least one dose of a mu opioid analgesic. In another embodiment, the invention provides a method for preventing, inhibiting or treating nausea and vomiting in a human, by administering a peripherally-restricted kappa opioid receptor agonist to the human, wherein the nausea and/or vomiting occurs within 24 hours after administration of at least one dose of a mu opioid analgesic. In a particular embodiment, the mu opioid analgesic is administered to treat, inhibit or prevent hard tissue pain, such as bone pain. The hard tissue pain may be due to a medical procedure. The medical procedure may be any medical procedure that causes hard tissue pain, such as, for instance and without limitation, a bunionectomy procedure.

In another embodiment, the invention provides a method for preventing, inhibiting or treating nausea and vomiting in a human, by administering a peripherally-restricted kappa opioid receptor agonist to the human, wherein the peripherally-restricted kappa opioid receptor agonist is administered prior to administration of a first dose of the mu opioid analgesic. In still another embodiment, the peripherally-restricted kappa opioid receptor agonist is co-administered with at least one dose of the mu opioid analgesic. In yet another embodiment, the peripherally-restricted kappa opioid receptor agonist is administered after administration of at least one dose of the mu opioid analgesic.

### Kappa Receptor Agonist CR845

CR845 is a peripherally-acting kappa opioid receptor agonist useful for treatment of both acute and chronic pain, and also has anti-inflammatory properties. The most advanced product candidate, I.V. CR845, has demonstrated significant pain relief and a favorable safety and tolerability profile in three Phase 2 clinical trials in patients with acute postoperative pain. Due to its selectivity for the kappa opioid receptor and ability to decrease mu opioid use, CR845 has demonstrated a consistent ability to decrease the acute opioid-related adverse events (AEs) of nausea and vomiting with no evidence of drug-related respiratory depression. CR845 has been administered to over 300 human subjects in Phase 1 and Phase 2 clinical trials as an intravenous infusion, short bolus or oral capsule and was safe and well tolerated in these clinical trials.

In standard preclinical pain models, CR845 successfully attenuated acute and chronic visceral, inflammatory and neuropathic pain in a dose-dependent manner (see Table 1, below). The analgesic effect of CR845 was recordable within 15 minutes post-administration and lasted for up to 18 hours following single-dose administration. CR845 also decreased the production and release of pro-inflammatory mediators, likely due to the direct activation of kappa opioid receptors expressed on immune cells that synthesize and secrete these substances.

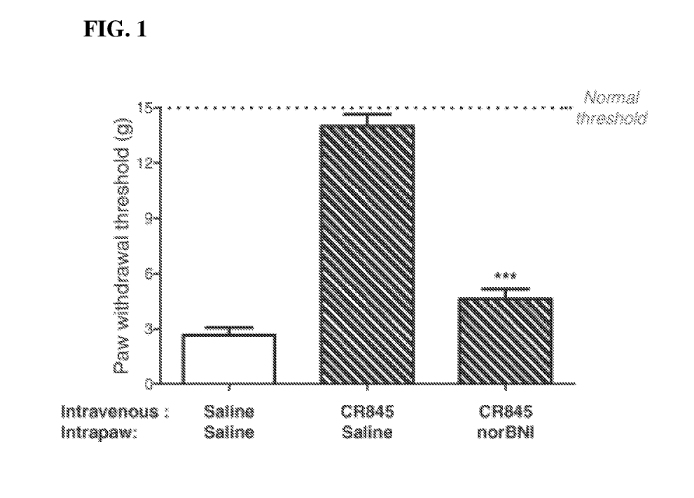

The peripheral mechanism of action of CR845 is supported preclinically by both biochemical in vitro assays and in vivo functional pharmacological studies. In pharmacokinetic studies, animals administered analgesic and supra-analgesic doses of CR845 exhibited no measurable concentrations of drug in extracted brain tissue indicating that the CNS was not the site of action for CR845. Moreover, in standard preclinical pain models, such as the “Chung Model” of neuropathic pain, the analgesic action of CR845 was blocked with kappa opioid receptor antagonists administered directly to the local site of injury, indicating a peripheral site of action for CR845 (FIG. 1). In the “Chung Model”, neuropathic pain is induced experimentally by ligating spinal nerves mediating sensation for a hind limb. This results in a type of neuropathic pain, referred to as allodynia. Experimental animals with allodynia exhibit a “paw withdrawal reflex” upon contact with a relatively thin filament on the injured site. Sets of different thickness filaments are used to test sensitivity, each of which is designed to produce a given force (in grams) upon bending after contact. By testing with these filaments, the minimum force to evoke a withdrawal response defines the paw withdrawal threshold. The nerve injury produces a marked reduction in paw withdrawal thresholds (increased sensitivity to force) in response to probing with the filaments. I.V. administration of CR845 reduces this neuropathic pain as demonstrated by a subsequent increase in the withdrawal threshold (see FIG. 1).

Administration of a low dose of the selective peripherally-acting kappa opioid receptor antagonist nor-binaltorphamine, or nor-BNI, into the plantar surface of the injured paw significantly reduces the effect of CR845, whereas injection of saline had no effect on the efficacy of CR845. Since nor-BNI was only able to block local peripheral kappa opioid receptors in this experiment, these results show that the effect of CR845 is a result of activation of kappa opioid receptors located at the peripheral site of injury rather than in the CNS.

### Intravenous CR845

CR845, in an injectable version of the most advanced kappa opioid receptor-based peripheral analgesic is designed to provide pain relief without stimulating mu opioid receptors and therefore without mu opioid-related side effects, such as nausea, vomiting, respiratory depression and euphoria. Intravenous CR845 has demonstrated efficacy and tolerability in three randomized, double-blind, placebo-controlled Phase 2 clinical trials in patients undergoing soft tissue (laparoscopic hysterectomy) and hard tissue (bunionectomy) surgery. In both the laparoscopic hysterectomy and bunionectomy clinical trials, CR845 administration resulted in statistically significant reductions in pain intensity, as measured by summed pain intensity differences, or SPID, which is the FDA-recommended acute pain endpoint: See below.

A Phase 2 multicenter, double-randomized, double-blind, placebo-controlled clinical trial (CLIN2002) was a conducted in 203 patients at 22 sites in the United States. The trial enrolled female patients, ages 21 to 65, scheduled for elective laparoscopic hysterectomy under general anesthesia. In this trial, patients were administered either placebo or one dose of 0.04 mg/kg I.V. CR845 preoperatively. Following surgery, if they were medically stable and had a pain intensity score ≧40 on a 100 point pain scale based on the visual analog scale, or VAS, they were re-randomized to receive either placebo or one dose of 0.04 mg/kg I.V. CR845. Efficacy was measured using time-specific 24 hour pain intensity differences. Pain intensity, or PI, was measured at various times by asking patients to rate their pain on a 100-point scale, where “0” is absence of pain and “100” is the worst possible pain. PID, or pain intensity difference, is the difference between the PI measured prior to treatment and at subsequent times of measurement. SPID, or the summed pain intensity difference, is the time-weighted sum of all of the PID scores, from the pretreatment level to a subsequent time of measurement, such as 24 hours after the pretreatment baseline pain measurement. Both PID and SPID are FDA-recognized endpoints for acute pain clinical trials. Additional endpoints included the amount of morphine consumption over 24 hours, time-specific total pain relief and patient global evaluation of study medication. Of the 203 patients that participated in the trial, 183 received a post operative dose; however, two subjects did not record baseline pain scores and were not included in calculated PID and SPID values. Accordingly, four treatment groups resulted from pre- and post-operative randomization:
(1) I.V. CR845 administered both preoperatively and postoperatively (CR845/CR845);
(2) placebo administered preoperatively and I.V. CR845 administered postoperatively (Placebo/CR845);
(3) I.V. CR845 administered preoperatively and placebo administered postoperatively (CR845/Placebo); and
(4) placebo administered both preoperatively and postoperatively (Placebo/Placebo).

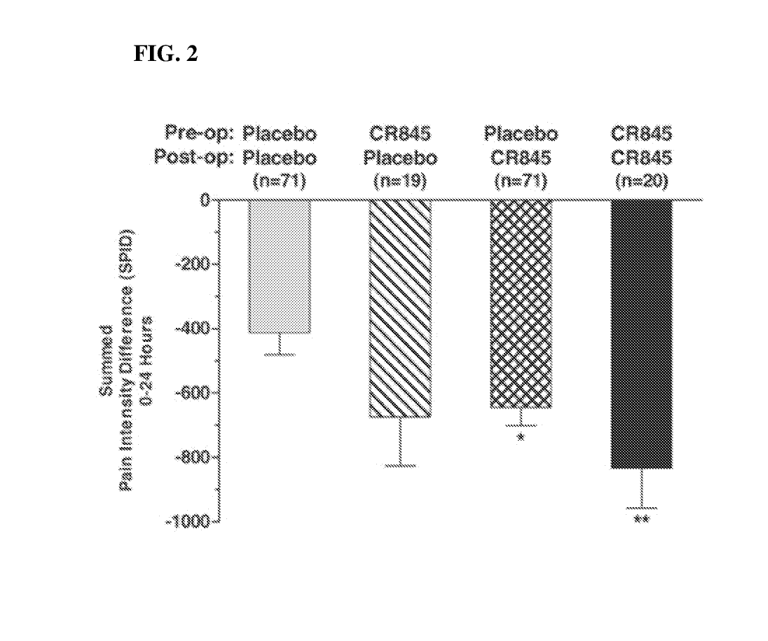

The CR845/CR845 group exhibited a statistically significant reduction in pain over a 24-hour time period, as indicated by an improvement in 0-24 hour mean SPID, compared to the Placebo/Placebo group (p≦0.01). The Placebo/CR845 group also exhibited a statistically significant improvement in 0-24 hour mean SPID compared to the Placebo/Placebo group (p≦0.05). The CR845/Placebo group exhibited an improved 0-24 hour mean SPID compared to the Placebo/Placebo group, but this difference did not reach statistical significance, which we believe was due to the small number of patients. FIG. 2 illustrates the 0-24 hour mean SPIDs of the four treatment groups listed above.

Similar observations were made for different time periods after treatment. For example, over the 0-4 hour time period, in the CR845/CR845 group, there was a statistically significant 3.5-fold improvement in mean SPID values compared to the Placebo/Placebo group (p≦0.05). In addition, over the 0-8, 0-12 and 0-16 time periods, patients in the Placebo/CR845 group also exhibited reduced pain intensity compared to the Placebo/Placebo group in a statistically significant manner (p≦0.05), based on improved SPID values.

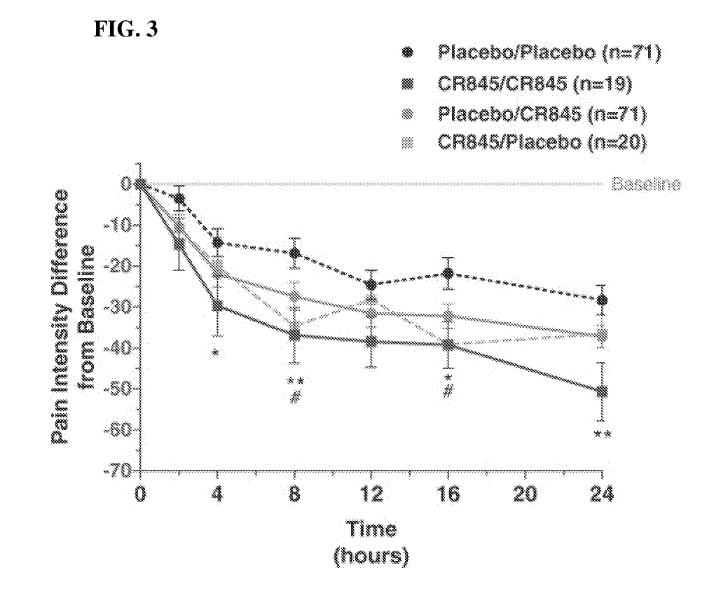

The mean PID from baseline at each time interval was numerically superior across all groups that received I.V. CR845 preoperatively and/or postoperatively relative to the Placebo/Placebo group. Compared to the Placebo/Placebo group, patients in the CR845/CR845 group exhibited an approximately 60% greater reduction in pain intensity at 24 hours (p≦0.01), as well as statistically significant improvements for the 0-4, 0-8 and 0-16 hour time intervals. Patients in the CR845/Placebo and Placebo/CR845 groups also exhibited statistically significant decreases in pain intensity for the 0-8 and 0-16 hour time intervals, compared to patients in the Placebo/Placebo group. FIG. 3 illustrates the PID relative to postoperative baseline in patients in the four treatment groups.

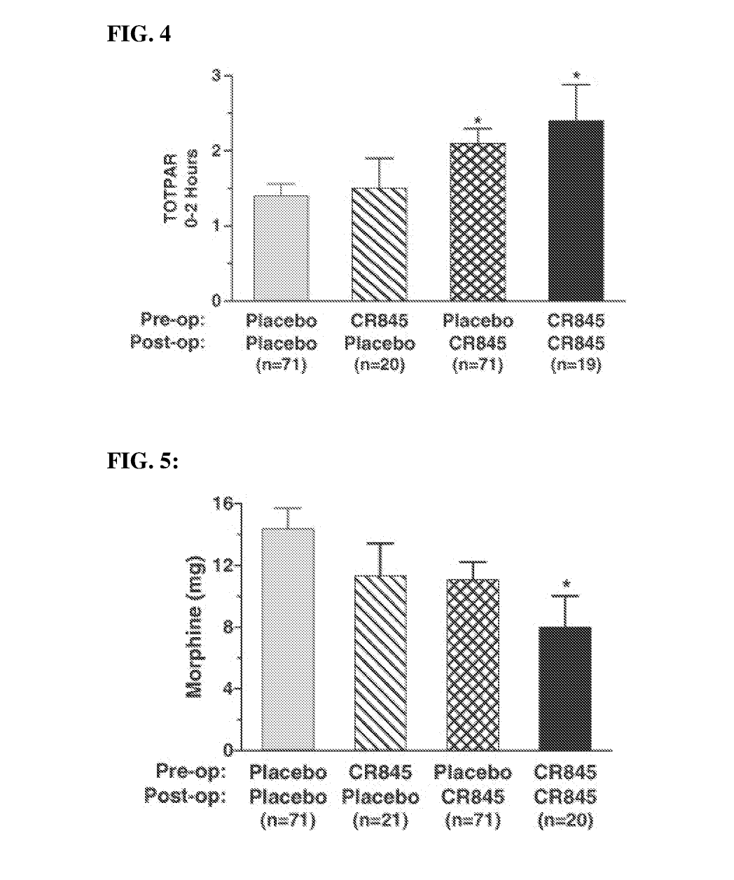

At the same time points at which pain intensity measurements were taken, patients' perceived pain relief scores were recorded using a 5 point subjective Likert scale (0-4), where zero corresponds to no relief and a score of four represents total relief. The “TOTPAR” score is calculated as the “total pain relief score”, which is a time-weighted sum of pain relief scores over any given time period following post operative treatment with CR845 or placebo. Mean TOTPAR scores were numerically superior across all intervals for the CR845/CR845 and Placebo/CR845 groups relative to the Placebo/Placebo group. The patients in the CR845/CR845 group and Placebo/CR845 exhibited statistically superior pain relief as compared to the Placebo/Placebo group within the first 2 hours following postoperative randomization, as indicated by increased mean TOTPAR₀₋₂ values (p≦0.05). FIG. 4 depicts the mean TOTPAR scores for the first 2 hour period for each of the four treatment groups listed above.

In the CR845/CR845 and Placebo/CR845 groups, there were also statistically significant improvements in reported pain relief for the 0-4, 2-4 and 0-8 hour time periods. In addition, the improvement in mean TOTPAR also reached statistical significance for the 0-12 hour interval for the CR845/CR845 group relative to the Placebo/Placebo group.

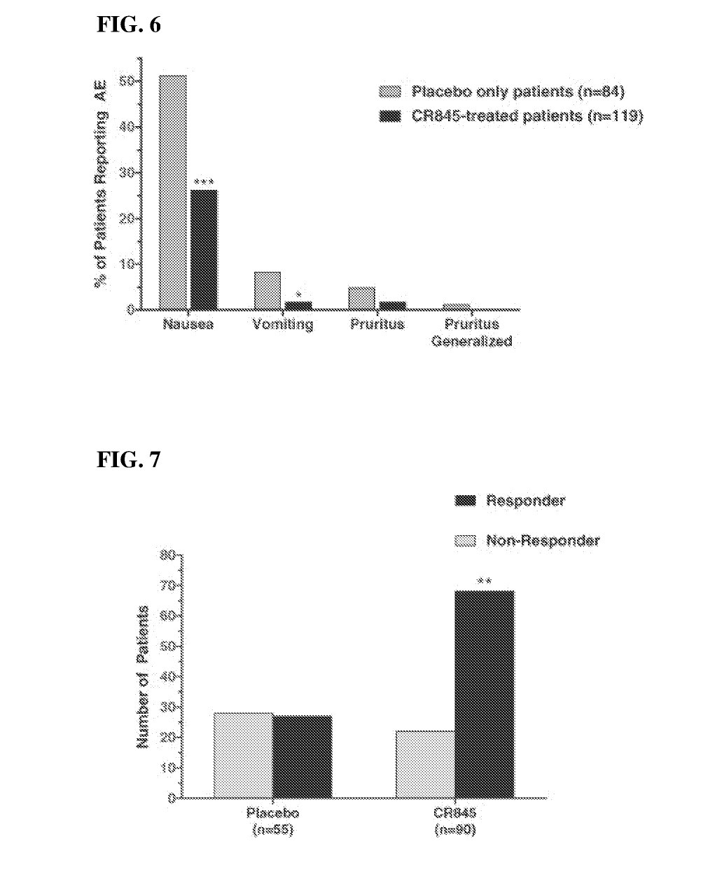

Intravenous morphine was available as rescue medication to all treatment groups upon patient request. Calculations of morphine consumption per treatment group in the 2-24 hour period, after patients leave the post-anesthesia care unit, or PACU, indicated that patients in the CR845/CR845 group used approximately 45% less morphine than those in the Placebo/Placebo group (p≦0.05) and patients in the Placebo/CR845 and CR845/Placebo groups used approximately 23% less morphine than those in the Placebo/Placebo group. FIG. 5 depicts the morphine usage in each of the treatment groups between hours 2-24. Concurrently with the observed reduction in morphine use, patients treated with I.V. CR845 exhibited a statistically significant lower incidence of opioid-related AEs through 24 hours after the start of the first infusion compared to patients who received only placebo. The incidence of nausea was reduced by approximately 50% (only 26.1% of patients administered CR845 experienced nausea as compared to 51.2% for placebo, p≦0.001) and the incidence of vomiting was reduced nearly 80% (only 1.7% of patients administered CR845 experienced vomiting, as compared to 8.3% for placebo, p=0.035). There was also less pruritus, or itching sensation, reported in patients treated with CR845 compared to placebo. FIG. 6 depicts the percentage of patients reporting opioid-related adverse events of nausea, vomiting and pruritus.

In addition to the reduction of opioid-related adverse events, a standard responder analysis indicated that a higher percentage of patients who received I.V. CR845 were characterized as “Responders” as compared to those receiving placebo (p=0.001). Responders included patients who rated their medication “Excellent” or “Very Good” and Non-Responders as those who rated their medication “Fair” or “Poor”. The lower overall pain intensity scores at the end of the study period for CR845-treated patients and the significant reduction in nausea and vomiting reported in these patients contributed to patients' greater satisfaction with I.V. CR845 treatment compared to placebo. FIG. 7 shows the number of Responder or Non-Responder patients in the I.V. CR845-treated patients compared to the patients receiving only placebo.

In this trial, intravenous administration of 0.04 mg/kg of I.V. CR845 pre- and/or post-operatively was safe and generally well tolerated. The placebo and CR845 treatment patient groups showed a similar overall incidence of treatment-emergent adverse events (TEAEs), the majority of which were mild to moderate in severity. The most frequent TEAEs, reported in 10% or more of total patients, were nausea, hypotension, flatulence, blood sodium increase, or hypernatremia, and headache. There were no apparent consistent differences between CR845 and placebo groups in clinical laboratory results, vital signs, electrocardiogram, or oxygen saturation results, with the exception of blood sodium increase, which was evident only in CR845 treatment groups (14% of total patients). The increase in blood sodium levels (hyper-natremia), observed in CR845 treatment groups was likely a result of the aquaretic effect of I.V. CR845 at this dose and the replacement of fluid loss with sodium-containing I.V. solutions, rather than water or low/no sodium-containing fluids. In subsequent trials, fluid replacement with water or I.V. solutions with low or no sodium was used with no evidence of hypernatremia.

### CR845 for Bunionectomy

Bunionectomy is a surgical procedure to remove a bunion, an enlargement of the joint at the base of the big toe and includes bone and soft tissue. The procedure typically results in intense pain requiring postoperative analgesic care, usually beginning with local anesthetic infusion and ongoing administration of a strong opioid, such as morphine or fentanyl, for several days after surgery during which the patient often suffers from nausea and vomiting.

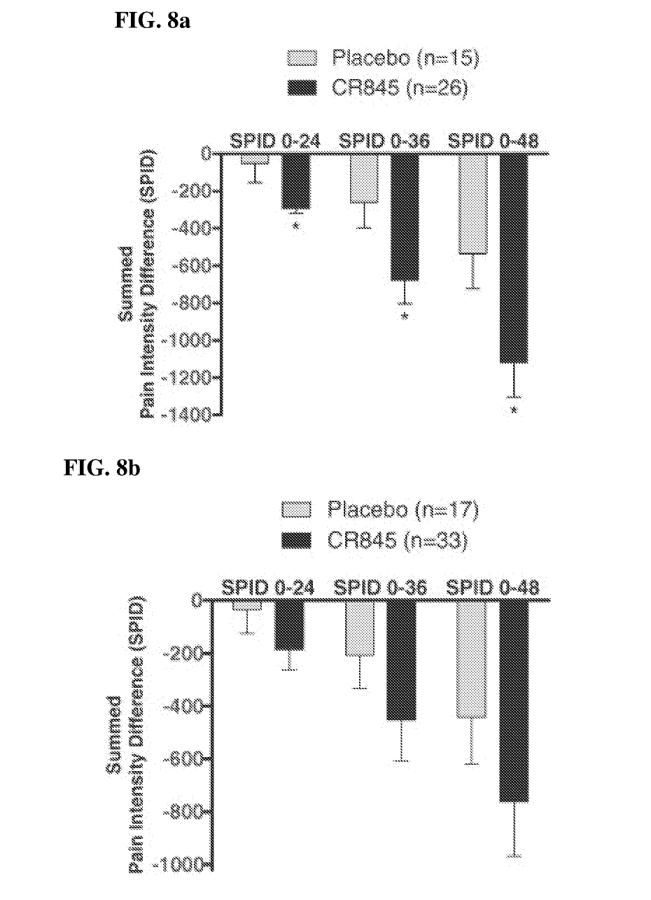

Clinical trial (CLIN2003) was a randomized, double-blind, placebo-controlled trial conducted in 51 patients following bunionectomy surgery at a single site in the U.S. The trial enrolled female and male patients, ages 18 years and older, scheduled for elective bunionectomy under regional anesthesia. Using a standard clinical trial protocol in which local anesthetic infusion was terminated on the day after surgery, patients were randomized into one of two treatment groups (CR845 or Placebo, in a 2:1 ratio) after reporting moderate-to-severe pain, defined as a pain intensity score≧40 on a 100-point pain scale. Patients randomized to receive I.V. CR845 were administered an I.V. injection at a dose of 0.005 mg/kg, and additional doses on an as-needed basis 30-60 minutes later, and then no more frequently than every 8 hours through a 48-hour dosing period. The results were analyzed separately for the per protocol population, or “Completers”, which includes only patients who completed the trial, and the modified Intent-to-Treat, or mITT, population, which includes Completers and all patients who discontinued the trial, or “non-Completers”. In the Completer group, CR845 treatment resulted in a statistically significant reduction in pain intensity compared to placebo, as measured by the SPID score over the initial 24 hour time period (SPID₀₋₂₄; p<0.05). This reduction in pain intensity after CR845 dosing was also statistically significant over a 36 hour time period (SPID₀₋₃₆, p<0.03), as well as over the entire two-day dosing period (SPID₀₋₄₈, p<0.03), compared to placebo-treated patients (see FIG. 8a). Numerical improvements in SPID scores in the CR845 group as compared to placebo were also evident across the same time periods when analyzing the mITT population of Completers together with non-Completers (see FIG. 8b).

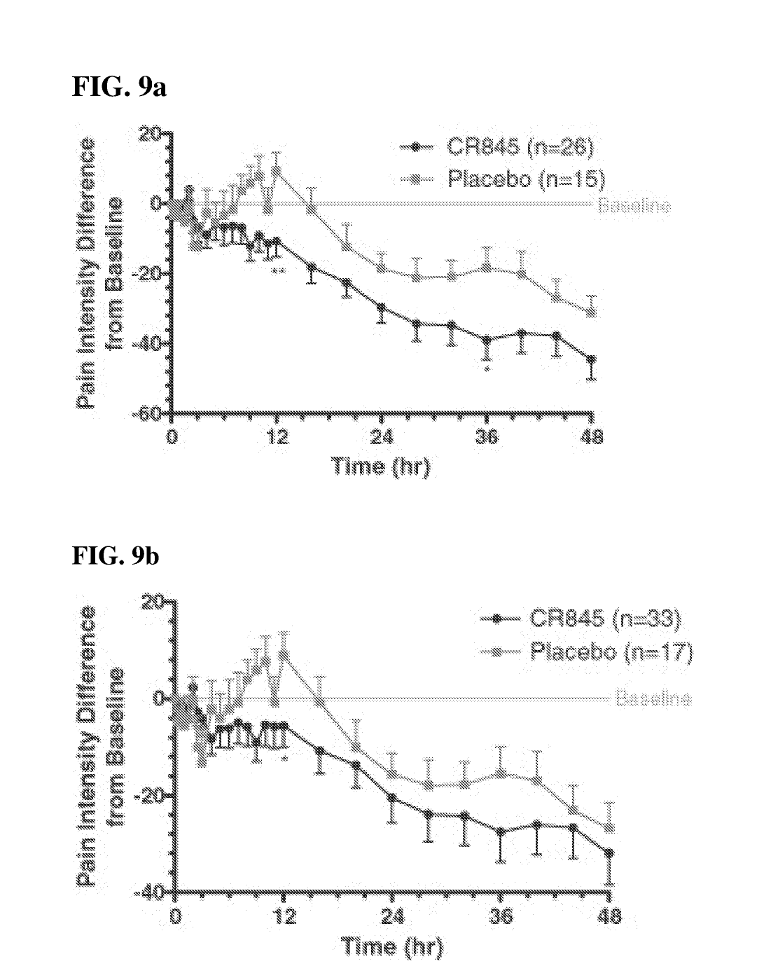

The Completer analysis is indicative of the actual efficacy of I.V. CR845 under conditions where patients are exposed to the drug as specified in the protocol, while the mITT analysis is indicative of the actual variability that will be encountered in the mITT populations. The understanding of this variability serves as the basis for determining the appropriate number of patients for enrollment in our Phase 3 clinical trials. In this trial, mean PID from baseline at each time interval was measured, and was numerically superior across the 48 hour trial period in the I.V. CR845 treatment group relative to the placebo group for both the Completer and mITT populations (see FIGS. 9a and 9b). Statistically significant reductions in pain intensity differences in the CR845 group versus placebo were evident in the 0-12 hour time interval for both the Completer and mITT populations (p≦0.01 and p≦0.05 respectively) and for the 0-36 hour time interval for the Completer populations (p≦0.05), consistent with the findings with the primary SPID endpoints.

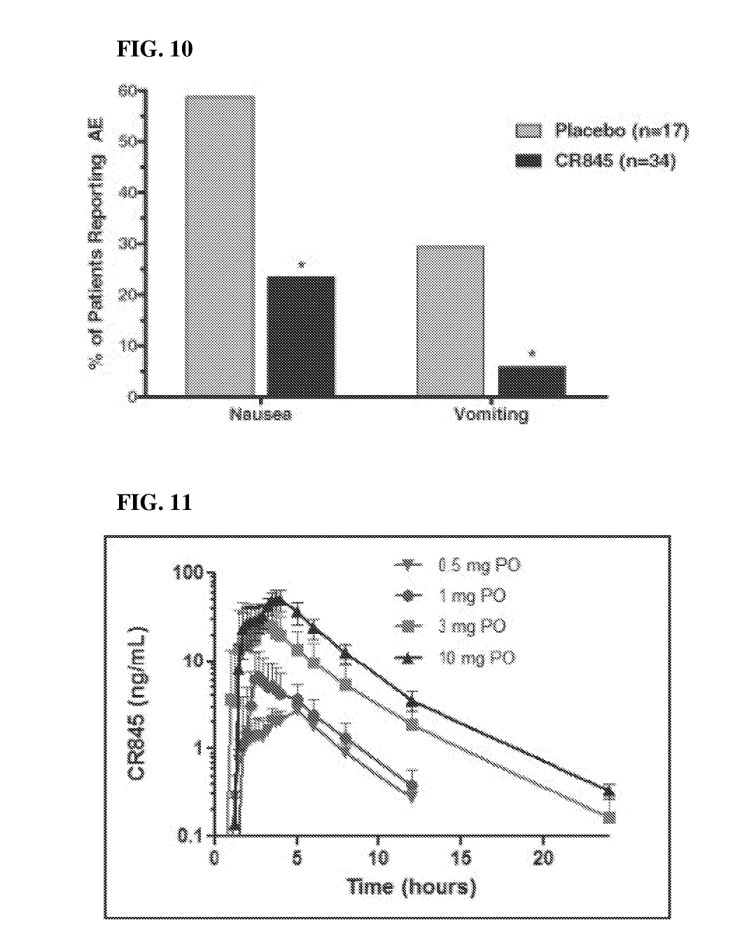

Fentanyl was available to both CR845 and placebo treatment groups upon patient request. While there was no difference in mean fentanyl use between the placebo and CR845 groups, the incidence of opioid-related AEs of nausea and vomiting was significantly reduced (by 60% and 80%, respectively; p≦0.05) in patients who received CR845 compared to placebo during the 48 hour period after randomization (see FIG. 10). Without wishing to be bound by theory, the ability of I.V. CR845 to reduce nausea and vomiting despite not meaningfully reducing fentanyl usage is believed to be due to a direct antiemetic effect resulting from its kappa opioid agonist mechanism of action. The ability to provide postsurgical analgesia and simultaneously reduce opioid-related side effects makes I.V. CR845 an attractive treatment option for postoperative patients and their physicians. In this bunionectomy trial, repeated intravenous administration of I.V. CR845 at a dose of 0.005 mg/kg was safe and generally well tolerated. The most frequent TEAEs (greater than 10%) observed in the CR845 treatment group were transient facial tingling and somnolence. Of the seven cases of somnolence reported, four were reported as “mild” and/or “related to drug” and three as “moderate” and/or “not related to drug”. The mean plasma sodium concentration in CR845-treated patients exhibited an approximately 3% rise over 24 hours from baseline levels, but was not outside the normal physiological range at either 24 or 48 hours post-CR845 administration. This lack of clinically significant hypernatremia was likely a result of both utilizing a lower dose of I.V. CR845 and replacing transient fluid loss with oral water or sodium-free intravenous fluid. In addition, consistent with our prior studies, there was no evidence of acute psychiatric side effects that were observed with prior-generation CNS-active kappa opioid agonists.

1. A method for preventing, inhibiting or treating nausea and/or vomiting in a mammalian subject, the method comprising administering an effective amount of a peripherally-restricted kappa opioid receptor agonist to the mammalian subject.

2. The method according to claim 1, wherein the peripherally-restricted kappa opioid receptor agonist comprises a peptide.

3. The method according to claim 1, wherein the peripherally-restricted kappa opioid receptor agonist comprises one or more D-amino acids.

4. The method according to claim 1, wherein the peripherally restricted kappa opioid receptor agonist comprises a synthetic peptide amide having the formula: 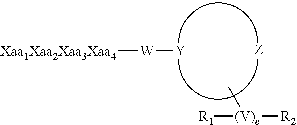
or a stereoisomer, mixture of stereoisomers, prodrug, pharmaceutically acceptable salt, hydrate, solvate, acid salt hydrate, N-oxide or isomorphic crystalline form thereof;
wherein
Xaa₁ is selected from the group consisting of (A)(A′)D-Phe, (A)(A′)(α-Me)D-Phe, D-Tyr, D-Tic, D-tert-leucine, D-neopentylglycine, D-phenylglycine, D-homophenylalanine, and β-(E)D-Ala, wherein each (A) and each (A′) are phenyl ring substituents independently selected from the group consisting of —H, —F, —Cl, —NO₂, —CH₃, —CF₃, —CN, and —CONH₂, and wherein each (E) is independently selected from the group consisting of cyclobutyl, cyclopentyl, cyclohexyl, pyridyl, thienyl and thiazolyl;
Xaa₂ is selected from the group consisting of (A)(A′)D-Phe, 3,4-dichloro-D-Phe, (A)(A′)(α-Me)D-Phe, D-1Nal, D-2Nal, D-Tyr, (E)D-Ala and D-Trp;
Xaa₃ is selected from the group consisting of D-Nle, D-Phe, (E)D-Ala, D-Leu, (α-Me)D-Leu, D-Hle, D-Val, and D-Met;
Xaa₄ is selected from the group consisting of (B)₂D-Arg, (B)₂D-Nar, (B)₂D-Har, ζ-(B)D-Hlys, D-Dap, ε-(B)D-Lys, ε-(B)₂-D-Lys, D-Amf, amidino-D-Amf, γ-(B)₂D-Dbu, δ-(B)₂α-(B′)D-Orn, D-2-amino-3(4-piperidyl)propionic acid, D-2-amino-3 (2-aminopyrrolidyl)propionic acid, D-α-amino-β-amidinopropionic acid, α-amino-4-piperidineacetic acid, cis-α,4-diaminocyclohexane acetic acid, trans-α,4-diaminocyclohexaneacetic acid, cis-α-amino-4-methylaminocyclo-hexane acetic acid, trans-α-amino-4-methylaminocyclohexane acetic acid, α-amino-1-amidino-4-piperidineacetic acid, cis-α-amino-4-guanidinocyclohexane acetic acid, and trans-α-amino-4-guanidinocyclohexane acetic acid, wherein each (B) is independently selected from the group consisting of H and C₁-C₄ alkyl, and (B′) is H or (α-Me);
W is selected from the group consisting of:
Null, provided that when W is null, Y is N;
—NH—(CH₂)_{b}— with b equal to zero, 1, 2, 3, 4, 5, or 6; and
—NH—(CH₂)_{c}—O— with c equal to 2, or 3, provided that Y is C;
the moiety 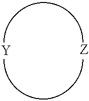
is an optionally substituted 4 to 8-membered heterocyclic ring moiety wherein all ring heteroatoms in said ring moiety are N; wherein Y and Z are each independently C or N; provided that when such ring moiety is a six, seven or eight-membered ring, Y and Z are separated by at least two ring atoms; and provided that when such ring moiety has a single ring heteroatom which is N, then such ring moiety is non-aromatic;
V is C₁-C₆ alkyl, and e is zero or 1, wherein when e is zero, then V is null and R₁ and R₂ are directly bonded to the same or different ring atoms;
wherein (i) R₁ is selected from the group consisting of —H, —OH, halo, —CF₃, —NH₂, —COOH, C₁-C₆ alkyl, C₁-C₆ alkoxy, amidino, C₁-C₆ alkyl-substituted amidino, aryl, optionally substituted heterocyclyl, Pro-amide, Pro, Gly, Ala, Val, Leu, Ile, Lys, Arg, Orn, Ser, Thr, —CN, —CONH₂, —COR′, —SO₂R′, —CONR′R″, —NHCOR′, OR′ and SO₂NR′R″; wherein said optionally substituted heterocyclyl is optionally singly or doubly substituted with substituents independently selected from the group consisting of C₁-C₆ alkyl, C₁-C₆ alkoxy, oxo, —OH, —Cl, —F, —NH₂, —NO₂, —CN, —COOH, and amidino; wherein R′ and R″ are each independently —H, C₁-C₈ alkyl, aryl, or heterocyclyl or R′ and R″ are combined to form a 4- to 8-membered ring, which ring is optionally singly or doubly substituted with substituents independently selected from the group consisting of C₁-C₆ alkyl, —C₁-C₆ alkoxy, —OH, —Cl, —F, —NH₂, —NO₂, —CN, —COOH and amidino; and R₂ is selected from the group consisting of —H, amidino, singly or doubly C₁-C₆ alkyl-substituted amidino, —CN, —CONH₂, —CONR′R″, —NHCOR′, —SO₂NR′R″ and —COOH; or
(ii) R₁ and R₂ taken together can form an optionally substituted 4- to 9-membered heterocyclic monocyclic or bicyclic ring moiety which is bonded to a single ring atom of the Y and Z-containing ring moiety; or
(iii) R₁ and R₂ taken together with a single ring atom of the Y and Z-containing ring moiety can form an optionally substituted 4- to 8-membered heterocyclic ring moiety to form a spiro structure; or
(iv) R₁ and R₂ taken together with two or more adjacent ring atoms of the Y and Z-containing ring moiety can form an optionally substituted 4- to 9-membered heterocyclic monocyclic or bicyclic ring moiety fused to the Y and Z-containing ring moiety;
wherein each of said optionally substituted 4-, 5-, 6,-, 7-, 8- and 9-membered heterocyclic ring moieties comprising R₁ and R₂ is optionally singly or doubly substituted with substituents independently selected from the group consisting of C₁-C₆ alkyl, C₁-C₆ alkoxy, optionally substituted phenyl, oxo, —OH, —Cl, —F, —NH₂, —NO₂, —CN, —COOH, and amidino;
provided that when the Y and Z-containing ring moiety is a six or seven membered ring having a single ring heteroatom and e is zero, then R₁ is not —OH, and R₁ and R₂ are not both —H;
and
provided further that when the Y and Z-containing ring moiety is a six membered ring having two ring heteroatoms, both Y and Z are N and W is null, then —(V)ₑR₁R₂ is attached to a ring atom other than Z; and if e is zero, then R₁ and R₂ are not both —H.

5. The method of claim 4, wherein the moiety: 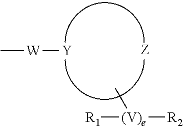
is selected from the group consisting of:

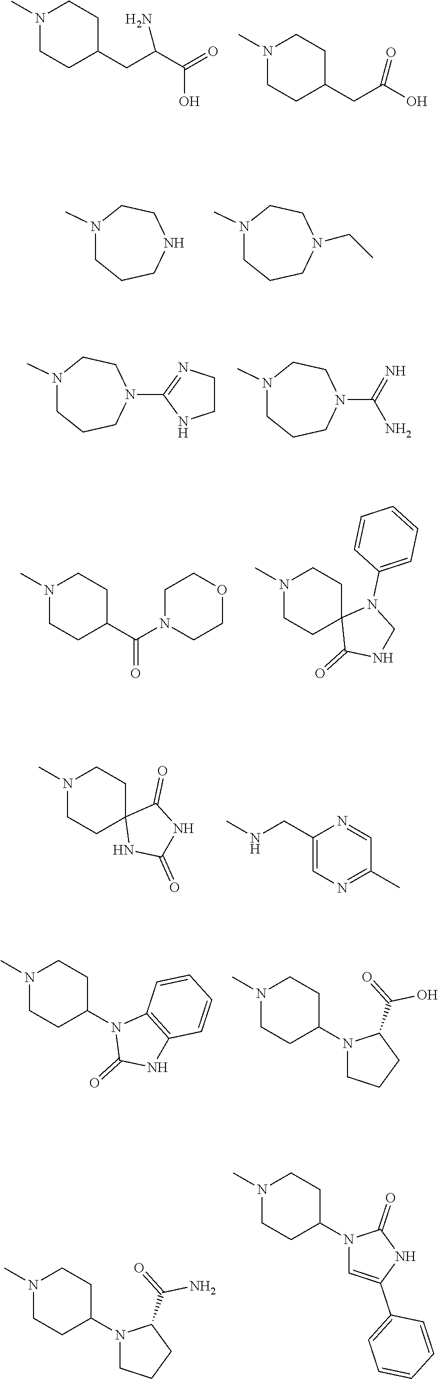

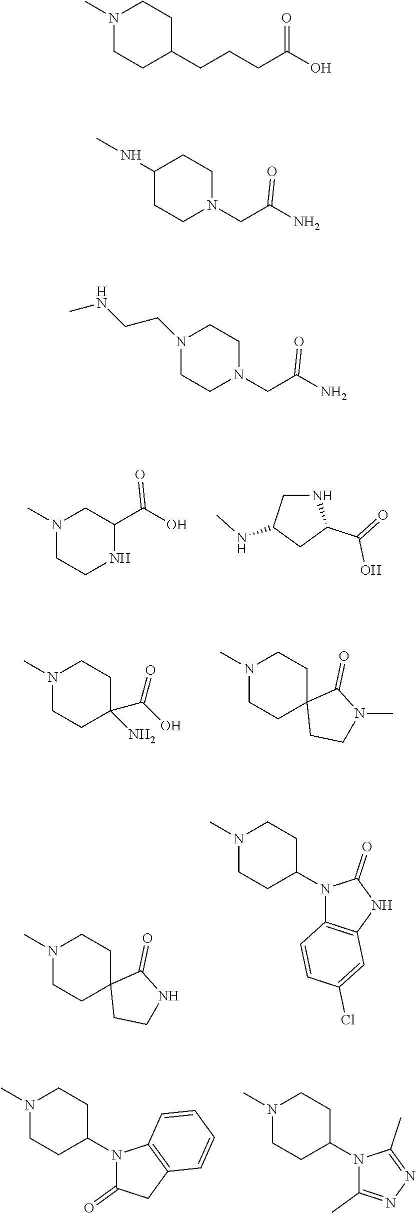

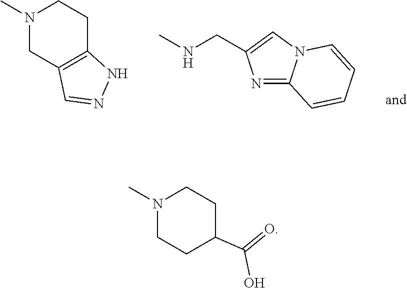

6. The method of claim 4, wherein the synthetic peptide amide has the structure: 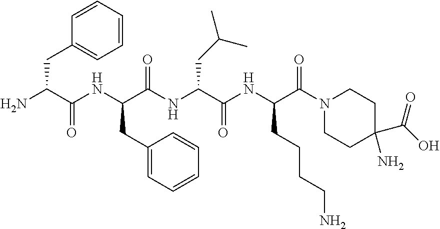 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]
D-Phe-D-Phe-D-Leu-D-Lys-[ω(4-aminopiperidine-4-carboxylic acid)]-OH.

7. The method of claim 6, wherein the mammalian subject is a human.

8. The method according to claim 1, wherein the peripherally-restricted kappa opioid receptor agonist is administered to the subject within 24 hours prior to, during, or within 24 hours after undergoing a medical procedure.

9. The method according to claim 8, wherein the medical procedure causes pain.

10. The method according to claim 6, wherein the peripherally-restricted kappa opioid receptor agonist is administered to the subject by a route of injection selected from the group consisting of subcutaneous injection, intravenous injection, intraperitoneal injection, intra-articular injection, and intramuscular injection.

11. The method according to claim 1, wherein the peripherally-restricted kappa opioid receptor agonist is a non-narcotic analgesic.

12. The method according to claim 1, wherein the peripherally-restricted kappa opioid receptor agonist is asimadoline (N-[(1S)-2-[(3S)-3 -hydroxypyrrolidin-1-yl]-1-phenylethyl]-N-methyl-2,2-diphenylacetamide).

13. The method according to claim 1, wherein the peripherally-restricted kappa opioid receptor agonist is nalfurafine ((2E)-N-[(5α,6β)-17-(cyclopropylmethyl)-3,14-dihydroxy-4,5-epoxymorphinan-6-yl]-3-(3-furyl)-N-methylacrylamide).

14. The method according to claim 1, wherein the mammal is a human.

15. The method according to claim 14, wherein the nausea and/or vomiting in the human occurs within 48 hours after administration of at least one dose of a mu opioid analgesic.

16. The method according to claim 15, wherein the mu opioid analgesic is administered to treat, inhibit or prevent hard tissue pain.

17. The method according to claim 16, wherein the hard tissue pain is bone pain.

18. The method according to claim 17, wherein the hard tissue pain is due to a medical procedure.

19. The method according to claim 15, wherein the peripherally-restricted kappa opioid receptor agonist is administered prior to administration of a first dose of the mu opioid analgesic.

20. The method according to claim 15, wherein the peripherally-restricted kappa opioid receptor agonist is administered after administration of at least one dose of the mu opioid analgesic.
